(19) United States
(12) Patent Application Publication
(10) Pub. No.: US 2008/0241878 A1
(43) Pub. Date: Oct. 2, 2008

(54) Method For Enzmatic Cross-Linking Of A Protein, Cross-Linked Protein Thus Obatained And Use Thereof

(76) Inventors: Johannes Wilhelmus Leonardus Boumans, Ouderkerk Aan De Amstel (NL); Ricardo Marinus Albertus Nagtegaal, Zeist (NL); Albertus Dunnewind, Wageningen (NL); Randolph Peter Happe, Zaandam (NL); Martin Anton Bos, Wageningen (NL); Merete Faergemand, Alleroed (DK); Peter Degn, Ega (DK)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/660,025

(22) PCT Filed: Aug. 10, 2005
(86) PCT No.: PCT/NL2005/000582

§ 371 Date: Jan. 15, 2008

(30) Foreign Application Priority Data
Aug. 12, 2004 (EP) 04077294.9

(51) Int. Cl. C12P 21/02; C07K 2/00; A23J 3/04; A23L 1/03

(52) U.S. Cl. 435/68.1; 530/402; 426/557; 426/586; 426/579; 426/582; 426/570; 426/565; 426/572; 426/564; 426/549; 426/589; 426/583; 426/592; 426/605; 426/641

(57) ABSTRACT

The present invention provides methods for enzymatic cross-linking of a protein comprising performing a reaction for enzymatic cross-linking of said protein in the presence of a phenolic saccharide, wherein said reaction comprises the step of forming protein-protein cross-links. The present invention further provides a cross-linked protein obtainable by a method according to the invention as well as a protein that is cross-linked by a phenolic saccharide and the use of such proteins in a foodstuff. The invention also provides a foodstuff comprising a cross-linked protein of the invention as well as the use of a phenolic saccharide as a cross-linking agent for proteins.

The present invention relates to a method for enzymatic cross-linking of a protein. The present invention further relates to cross-linked proteins thus obtained and to the use of such cross-linked proteins in foodstuffs.

Proteins may be cross-linked in order to improve their functional properties, such as water binding and gelation properties, as well as the properties such as the viscosity that they may provide to fluids. The principle use of cross-linked proteins in foodstuffs is related to improving the quality of the end product.

The cross-linking of proteins may be performed chemically, but is preferably performed enzymatically for use in foodstuffs, for example by using transglutaminase (Nielsen, Food Biotechnology (1995) 9:119-156) or members of the multi-copper oxidase family, such as laccase and bilirubin oxidase (EP 0 947 142). A disadvantage of protein cross-linking by using these enzymes is, however, that such protein cross-linking reactions can only successfully be performed with proteins that contain reactive groups that are sufficiently available to the enzymes. Examples of such proteins are casein, gelatin and wheat gluten. Globular proteins, on the other hand, have a structure that makes their reactive groups insufficiently accessible for enzymatic protein cross-linking. Examples of globular proteins are soy protein, β-lactoglobulin, α-lactalbumin, bovine serum albumin and ovalbumin.

There are basically two methods for obtaining foodstuffs with cross-linked proteins. Either proteins are cross-linked and subsequently added to the foodstuff, or proteins in the foodstuff are cross-linked by an enzyme which is added to the foodstuff itself or to the food-producing system, therefore added in situ.

In order to improve the accessibility of globular proteins for enzymatic cross-linking, methods are available by which the structure of globular proteins may be changed. For example, the structure of β-lactoglobulin may be modified by treatment of the protein with dithiotreitol (DTT). Upon such treatment, the modified protein can be cross-linked using transglutaminase. However, use of modifying compounds such as DTT is undesired, as it is not allowed to use such compounds in foodstuffs due to their inherent protein-modifying activity.

Several studies have shown that the action of various oxidoreductases can induce the formation of cross-links in proteins (Matheis and Whitaker (1987) J. Food Biochem. 11, 309; Feeney and Whitaker (1988) Am. Assoc. Cereal Chem., St. Paul, Minn., 21-46, Singh (1991) Trends Food Sci. Technol. 2, 196). Especially the cross-linking of various proteins with peroxidase (EC 1.11.1.7) (POD) has been investigated in some detail (Gross and Sizer (1959) J. Biol. Chem. 234, 1611; Aeschbach et al. (1976) Biochim. Biophys. Acta 439, 292; Stahmann et al. (1977) Biopolymers 16, 1307; Matheis and Whitaker (1984b) J. Prot. Chem. 3, 35). Horseradish POD could induce cross-linking of some proteins in the presence of hydrogen peroxide and a low molecular weight hydrogen donor yielding various oligo- and polymers, when incubated with e.g. ovalbumin, beta-LG and BSA (Stahmann et al. (1977) Biopolymers 16, 1307). Several proteins, e.g. casein, lysozyme and soy bean protein, were cross-linked using POD/hydrogen peroxide and oligo- and polymers were detected using SDS-PAGE {Matheis and Whitaker (1984b) J. Prot. Chem. 3, 35}. Casein has also been cross-linked using polyphenol oxidase (EC 1.11.18.1) in the presence of a low molecular weight phenolic compound (caffeic acid) {Hurrell et al., 1982}.

POD has a wide specificity towards electron donors in the oxidation process but specifically uses hydrogen peroxide as electron acceptor, when phenolic compounds or amines are the electron donors {Matheis and Whitaker, 1984a}. In proteins, POD mainly oxidises tyrosine and possibly cysteine and tryptophan {Sizer, 1953}. Evidence of the formation of tyrosine cross-links by POD and the ‘natural’ occurrence of dityrosine in proteins have been known for long {Sizer, 1953, Andersen, 1964} and the elasticity and insolubility of e.g. resilin, elastin and collagen have been ascribed to the presence of such cross-links {Andersen, 1964, Matheis and Whitaker, 1984a}. The oxidative formation of tyrosine cross-links in proteins with POD is thought to proceed through a free radical mechanism (Matheis and Whitaker (1984a) J. Food Biochem. 8, 137).

EP 0 947 142 discloses a method for cross-linking protein which uses a multi-copper oxidase. It is mentioned that various polyphenols may be added as a mediator which accelerates the reaction of the multi-copper oxidase.

EP 1 169 922 discloses a method of cross-linking a protein or peptide and a phenolic polymer or oligomer having substituents derived from carboxylic acids containing hydroxyl substituted phenyl groups by means of an enzyme and an oxidizing agent suitable for the enzyme in a solvent. The method comprises reacting a mixture of protein or peptide, oxidizing agent, enzyme and polymer or oligomer in the solvent, wherein the method is controlled such that in the mixture the ratio of target amino groups in the protein or peptide to hydroxyl substituted phenyl groups in the polymer or oligomer is more than equimolar. It is disclosed that predominantly hetero-cross-linking of protein and polymer occurs.

WO 03/007733 discloses a composition comprising a hydrocolloid, and an enzyme, wherein the enzyme is a cross-linking enzyme. The preferred enzyme is disclosed to be transglutaminase. As examples of hydrocolloids are mentioned molecules or polymolecular particles which are dispersed/dispersible in water or an aqueous solution such as e.g. polysaccharides. Examples of hydrocolloids include carrageenan, starch, pectin, guar gum, alginate, locust bean gum (LBG), gellan, xanthan, carboxy-methyl-cellulose (CIVIC), guar gum, acacia gum. The use of the hydrocolloids in combination with the enzymatic cross-linking peptide is described to improve gelation.

Although several methods for enzyme-aided protein cross-linking have been described, there is still a need for methods that alleviate some of the problems associated with the known methods such as costs, usefulness for globular proteins especially in their native state, reaction speed and usefulness for food grade products.

It is the aim of the present invention to provide a method for enzymatic cross-linking of protein by forming protein-protein cross-links.

Yet a further aim of the invention is to provide a method wherein the rate of enzymatic protein cross-linking is enhanced.

It is a further aim of the present invention to provide for a method of cross-linking that supports the enzymatic cross-linking of globular proteins. It is a further aim of the present invention to provide for a method of enzymatic cross-linking of globular proteins, which results in products that may be used in foodstuffs or wherein at least the use of non-food-grade structure-modifying reagents is prevented.

It has now surprisingly been found that these aims may be reached by performing an enzymatic cross-linking reaction in the presence of a phenolic saccharide. This finding may advantageously be used to improve the prior art methods for enzymatic cross-linking of proteins.

As yet, the precise mechanism of how the phenolic saccharide brings about the cross-linking of the protein is unknown. Without wishing to be bound by any theory, it is believed that the phenolic saccharide has a catalytic or cofactor-like effect that causes the formation of the desired protein-protein cross-links. Alternatively, the phenolic saccharide may have a mediating and/or enhancing and/or accelerating effect on the formation of the desired protein-protein cross-links. In a further alternative the phenolic saccharide may act as a linking or bridging molecule in the same way as a chemical cross-linking agent such as glutaric aldehyde.

In a first aspect, the present invention provides a method for enzymatic cross-linking of a protein comprising performing a reaction for enzymatic cross-linking of said protein in the presence of a phenolic saccharide, wherein said reaction comprises the step of forming protein-protein cross-links. Preferably, the method of the invention is performed in the presence of a phenolic saccharide which enhances the rate of a protein-protein cross-linking reaction. In a further aspect the method of the invention is performed in the presence of a phenolic saccharide which enhances the rate of a protein-protein cross-linking obtained compared to the rate obtained in the presence of enzyme alone. In yet a further aspect the method of the invention is performed in the presence of a phenolic saccharide which enhances the rate of a protein-protein cross-linking obtained compared to the rate obtained in the presence of enzyme and ferulic acid.

The method of the present invention may be used to cross-link any type of protein, including globular proteins. Specifically, however, a method of the present invention relates to the enzymatic cross-linking of a globular protein. The present method does not require the modification of the structure of the globular proteins, such as for instance by the removal of calcium from such proteins in order to modify their structure. Therefore, a method of the present invention preferably relates to the enzymatic cross-linking of a structurally unmodified globular protein.

The method of the present invention preferably relates to globular food proteins. Therefore, important food proteins such as whey proteins may now be cross-linked without the disadvantage of requiring the use of structure-modifying reagents that are incompatible with food-grade products. As a result, the method may be used to produce proteins that are exceptionally suitable for use in foodstuffs.

The term “phenolic saccharide” as used in the context of the present invention means a saccharide comprising one or more phenolic substituents.

In principle, any phenolic saccharide or combination of phenolic saccharides may be used in aspects of the present invention. The phenolic saccharide may be in the form of a monosaccharide, an oligosaccharide (including disaccharides) or a polysaccharide. In preferred embodiments of aspects of the present invention, the phenolic saccharide is a phenolic monosaccharide. In other preferred embodiments, the phenolic saccharide is a phenolic polysaccharide, even more preferably a phenolic polysaccharide wherein the saccharide chain is hydrolysed such as to form shorter fragments. In most preferred embodiments, the phenolic saccharide is a phenolic oligosaccharide, preferably derived from a hydrolysed phenolic polysaccharide. If desired, combinations of various phenolic saccharides may be employed in aspects of the invention.

The phenolic saccharide used in aspects of the present invention may be of natural or synthetic origin. Preferably, the phenolic saccharides are obtained from natural sources, such as plant sources. Such phenolic saccharides are potentially cheap and harmless ingredients. Phenolic saccharides are widely distributed in the plant kingdom and occur in fruits, leaves and other tissues of predominantly dicotyledonous plants. They may be extracted from a variety of natural sources such as green coffee beans (e.g., Arabica, Robusta and Liberica); leaves of Ilex paraguariensis, pome fruits (e.g., apples and pears); stone fruits (e.g., cherries and plums); berry fruits (e.g., strawberry, raspberry and blackberry); citrus fruits; brassica vegetables (e.g., kale, cabbage and brussels sprouts); Solanaceae (e.g., potato tubers, tomatoes, and aubergines); Asteraceae (e.g., chicoryroot and artichokes); Chenopodiaceae (e.g., beet, sugar beet) and a variety of other miscellaneous vegetables.

Examples of suitable phenolic polysaccharides are polysaccharides such as arabinoxylans, which contain phenolic substituents derived from cinnamic acid. Arabinoxylans may for instance be obtained from cereals. Another example of a suitable phenolic polysaccharide is pectin, which contain phenolic substituents derived from cinnamic acid. Such pectin may for instance be obtained from Chenopodiaceae such as beet, preferably sugar beet by methods known in the art. Sugar beet pectin is one of the few polysaccharides which contain ferulic acid. It is attached to the 0-2 position of (1,5)-linked arabinose residues in the arabinan side-chains and it can also be found to be attached to the 0-6 position of galactose residues in (1,4)-linked galactans. The number of substituents is preferably in the range of 1-50 ferulic acid residues.

Preferably, phenolic saccharide is selected from the group consisting of hydrolyzed arabinoxylans, hydrolyzed pectins such as hydrolysed sugar beet pectin, p-coumaroylglucose, feruloylglucose and feruloylarabinose. More preferred, the phenolic saccharide is selected from the group consisting of hydrolyzed sugar beet pectin and feruloylarabinose. It is apparent to a person skilled in the art that only a fraction of the molecules of hydrolysed polymeric sugar such as hydrolysed sugar beet pectin would carry ε ferulic acid residue. For the purpose of the present invention, the term phenolic saccharide in relation to hydrolysates means the entire population of molecules formed by hydrolysis.

The best cross-linking results are obtained when the molecular weight of the phenolic saccharide is relatively small. Preferably, the phenolic saccharide comprises a saccharide chain length of 1-20 sugar residues, wherein sugar residue is to be understood as a monosaccharide moiety (said chain length of the phenolic saccharide is herein also indicated by the term “degree of polymerization” [DP]). More preferably, the phenolic saccharide comprises a saccharide chain length of 1-9, more preferably of 1-5 sugar residues. Phenolic polysaccharides are thereto preferably hydrolyzed into smaller fragments, preferably of the above described preferred saccharide chain lengths, in order to be used in aspects of the present invention. Preferably, enzymatic hydrolysis is applied to acquire the desired saccharide chain length. Enzymatic hydrolysis of polysaccharides is well known in the art. Suitable enzymes include for instance exo- and endo-carbohydrases (hydrolases) and glycosidases, including pectinase, mannanase, cellulose, laminarinase (beta-glucanase), xylanase, cellulase, amylase, arabinase, galactanase and polygalacturonase. For the hydrolysis of for instance pectin, driselase, a multicomponent fungal enzyme preparation from a Basidiomycetes sp., may suitably be used. Reaction conditions can be used as indicated by the manufacturer.

It should be understood that the DP value is in some instances, especially in the case of hydrolyzed polysaccharides, a mean value for the degree of polymerization of the oligomers. Aspects of the present invention therefore preferably relate to phenolic saccharides with a DP less than 10, more preferably less than 9, even more preferably less than 8, still more preferably less than 7, and yet still more preferably less than 6, 5, 4, or 3.

When the phenolic saccharide is isolated from natural sources and hydrolyzed thereafter, the ester linkage by which the phenolic substituent is linked to the saccharide is preferably maintained during the hydrolysis reaction. Therefore, enzymes used in the purification of phenolic saccharide of the cinnamic type from polysaccharides are preferably free from ferulic acid esterase activity.

Many different phenolic substituents are suitable as phenolic group in phenolic saccharides used in aspects of the present invention. The phenolic substituents may suitably be derived from benzoic acid. Alternatively, the phenolic substituents may suitably be derived from cinnamic acid. p-Coumaric acid (p-hydroxycinnamic acid), ferulic acid (3-methoxy-4-hydroxycinnamic acid), caffeic acid (3-(3,4-dihydroxyphenyl)-2-propenoic acid) and sinapic acid (3,5-dimethoxy-4-hydroxycinnamic acid), for instance, are very suitable cinnamic acid-derived phenolic substituents. In fruits and vegetables, these hydroxycinnamic acids are widely present in derivatized form. Three glucosylated forms of these hydroxycinnamic acids are known to exist: the 3-O- and 4-O-glucosides and the glucose ester, all of which may be used in aspects of the present invention. The most common derivatives are esters with D-quinic acid and D-glucose and their abundance varies with plant species. p-Coumaroylglucose and feruloylglucose may for instance be found in apple, plum and a number of berries, like strawberry, raspberry and blackberry. Furthermore, O-glucosides of caffeic, coumaric and ferulic acid occur in berries. Different wheat gluten fractions also comprise a significant portion of low molecular weight phenolic saccharides, primarily phenolic sugars (mono- and disaccharides), mainly containing ferulic acid and sinapic acid.

The phenolic saccharide used in aspects of the present invention may also be synthesized chemically. In principle any method for synthesizing a phenolic saccharide useful in aspects of the present invention may be employed. Such methods are well known in the art. For instance, the phenolic saccharide may be prepared enzymatically by coupling a phenolic group to a saccharide. Ester linkages to phenolic carboxylic acids, such as cinnamic acids, may for instance also be synthesized by non-enzymatic methods known in the art. Sugars which contain acid groups, such as galacturonic acid or glucuronic acid, or oligomers or polymers which contain acid groups, such as pectin or carboxymethylcellulose and hydrolysates thereof, can be esterified with polyhydric phenolic substances, e.g. ferulic alcohol or sinapyl alcohol.

### Protein

The term “protein” as used in relation to the various aspects of the present invention relates to any type of protein including fibrous proteins and globular proteins, preferably globular proteins, more preferably globular food proteins. “Globular proteins” is used herein in its art-recognized meaning and includes proteins that have a globular domain. Preferably, however, aspects of the present invention relate to proteins that are strictly globular, i.e. those that can essentially not be cross-linked without the use of structure-modifying agents. Examples of globular proteins include soy protein, conalbumin, bovine serum albumin (BSA), hemoglobin, ovalbumin, α-chymotrypsinogen A, α-chymotrypsin, trypsin, trypsinogen, β-lactoglobulin, myoglobin, α-lactalbumin, lysozyme, ribonuclease A, cytochrome c, etc. In much preferred embodiments of aspects of the present invention, the globular protein is a food protein, even more preferably whey protein isolate (WPI), most preferably the globular protein is selected from the group consisting of soy protein, β-lactoglobulin, α-lactalbumin, bovine serum albumin and ovalbumin. In a further preferred aspect the globular protein is selected from the group consisting of β-lactoglobulin, α-lactalbumin, and bovine serum albumin. The protein may be of vegetable or animal origin, such as for instance egg protein, milk protein, gelatin, collagen, serum albumin, or may be derived from micro-organisms or algae. Further, as the protein used in the present invention, protein partially cleaved by a protease, etc., synthetic peptides and various proteins chemically modified can be used as the substrate in the enzymatic cross-linking reaction, in addition to those described above.

Aspects of the present invention may relate to a single type of protein, two different proteins or a combination of various different proteins that are (to be) cross-linked. Also natural proteins may be combined with synthetic peptides or with hybrid materials, such as polymers comprising peptide as well as glycosidic bonds and further comprising cross-linkable reactive groups. Alternatively, such hybrid materials may be used as the substrate material that is to be cross-linked by a method of the present invention.

### Cross-Linking Oxidase Enzyme

In performing a reaction for enzymatic cross-linking of a protein according to a method of the present invention any cross-linking oxidase enzyme can in principle be used. In some cases it may be appropriate to employ two or more different enzymes in the method according to the invention. Suitable enzymes include members of the multi-copper oxidase family, such as laccase and bilirubin oxidase. Cross-linking enzymes which can be used in the present invention are, inter alia, but not exclusively, polyphenol oxidases, alone as well as in combination. Preferably, as cross-linking enzyme, laccase (EC 1.10.3.2) is used. Examples of other useful, phenol-oxidizing oxidases are catechol oxidases, peroxidases and tyrosinases. In case peroxidases are used, the oxidizing agent is preferably hydrogen peroxide, methylperoxide, ethyloperoxide and/or sodiumperborate.

Other oxidases, such as various phenol oxidizing enzymes may be more suited for food purposes than the H₂O₂ requiring peroxidase. As used herein, the term “phenol oxidizing enzyme” refers to those enzymes which catalyze redox reactions and are specific for molecular oxygen and/or hydrogen peroxide as the electron acceptor. The phenol oxidizing enzymes of the present invention function by catalyzing redox reactions, i.e., the transfer of electrons from an electron donor (usually a phenolic compound) to molecular oxygen and/or hydrogen peroxide (which acts as an electron acceptor) which is reduced to water. Examples of such enzymes are laccases (EC 1.10.3.2), bilirubin oxidases (EC 1.3.3.5), phenol oxidases (EC 1.14.18.1) and catechol oxidases (EC 1.10.3.1).

Laccase uses oxygen as electron acceptor and is therefore highly suited for food purposes. Laccase primarily oxidises diphenols as electron donors but can also oxidize monophenols such as e.g. ferulic acid, yielding reactive quinines e.g. semi quinons as primary oxidation products {Yaropolov et al., 1994}. Reactive quinones may condense with each other or may react with other compounds. In proteins the oxidatively generated quinones are believed to e.g. initiate deamidation of the epsilon-amino group of lysine residues to give a peptide bound alpha-aminoadipic-delta-semialdehyde, which can then react to form cross-links via aldol condensation or aldimine condensation. Quinones may also react with the sulfhydryl group of cysteine, with the N-terminal amino group or with tyrosine {Hurrell et al., 1982, Kuninori et al., 1978}, thus a large number of non-enzymatic reactions may proceed after the enzymatic formation of reactive quinones.

Monoamine oxidase (MAO) mainly oxidises primary amines to yield aldehydes {Tipton, 1971} although the enzyme may also oxidise some secondary and tertiary amines {Yasunobu and Gomes, 1971}. Thus the primary oxidation products of this enzyme on lysine residues, i.e. aldehydes, may initiate some of the same reactions as the non-enzymatic reactions initiated by the quinones formed from the action of laccase.

In one aspect of the invention the cross-linking enzyme is laccase. In another aspect of the invention the cross-linking enzyme is laccase and the phenolic saccharide is a ferulic acid-containing oligosaccharide or monosaccharide, preferably hydrolyzed sugar beet pectin and feruloylarabinose.

### Enzymatic Cross-Linking Reaction

The enzymatic cross-linking reaction of a method according to the invention may be performed during the production of a foodstuff or protein-comprising product (e.g. in situ) in order to improve the properties of the proteins comprised therein, but the reaction is preferably performed in a separate reaction mixture. A reaction mixture for carrying out an enzymatic cross-linking reaction according to the invention can be prepared by suspending or diluting, preferably by dissolving the protein that is to be cross-linked, in a solvent. Preferably, the diluent or solvent is water.

An enzymatic cross-linking reaction of protein according to the invention can be carried out in a paste, a slurry, a dispersion or in a solution of the protein. Depending on the enzyme used for cross-linking, and the desired extent of cross-linking of the protein, the skilled person can adjust the reaction conditions, optimize them and deploy particular auxiliary substances to that end. For instance, the reaction period can be prolonged with the object of increasing the extent of cross-linking and thereby the property of the cross-linked protein to be achieved.

A reaction mixture for performing the enzymatic cross-linking reaction of the invention comprises a number of components. The order in which the different components are added to the reaction mixture is not limitative. A reaction mixture comprises at least a diluent or solvent, one or more proteins to be cross-linked (the target protein), one or more cross-linking enzymes and one or more phenolic saccharides.

The reaction mixture may comprise a single type of protein or two different proteins or a combination of various different proteins that are to be cross-linked.

A suitable amount of protein which is used in a reaction mixture according to the invention is between 1 and 99% by weight of protein, based on the weight of the reaction mixture. Preferably, between 1 and 50% by weight of protein is used. More preferably, an amount of protein between 1 and 20% or even between 2 and 10% by weight is used.

Buffering substances may optionally be added to a reaction mixture for carrying out a cross-linking reaction according to the invention in order to maintain the acidity thereof. The optimal pH depends mainly on the enzyme used for carrying out the cross-linking reaction. Preferably, the pH of the reaction mixture is in a range between approximately 3 and approximately 10, such as between 3 and 9. More preferably, the pH is between approximately 4 and approximately 8, still more preferably, the pH is between approximately 5 and 7, most preferably in the range of from approximately 5 to approximately 6.

The presence of emulsifiers and surface active agents such as stabilizers, and optionally different additives in the reaction mixture can promote the cross-linking reaction since they may keep the enzymes in an active form.

The reaction mixture further comprises a phenolic saccharide. A suitable amount of phenolic saccharide, which is used in a reaction mixture according to the invention, is between 0.05 and 99% by weight of phenolic saccharide, based on the weight of the reaction mixture. Preferably, between 0.1 and 20% by weight of phenolic saccharide is used, such as between 0.1 and 10% by weight. More preferably, an amount of phenolic saccharide of about 2 to 5% by weight is used. In general, the smaller amounts will be adequate when low molecular weight phenolic saccharides are used, whereas the higher amounts may be required when a hydrolysate is used which contains only a small fraction of the phenolic saccharide.

The reaction mixture further comprises a cross-linking enzyme, or a combination of cross-linking enzymes. In one aspect of the invention the phenolic saccharide, such as hydrolysed sugar beet pectin, is added to the reaction mixture in one step (batch-method).

As different cross-linking enzymes cross-link different amino acids, in view of this, modification of the reaction conditions is possible. In principle, the reaction conditions during the cross-linking reaction are selected such that an optimal cross-linking can take place. These reaction conditions comprise conditions such as concentration of the cross-linking enzyme, temperature, duration of time of the reaction, pH, salt concentration, protein concentration phenolic saccharide concentration and the presence of optional auxiliary substances. The optimal reaction conditions during a cross-linking reaction of a protein can differ for different cross-linking enzymes. For instance, in a cross-linking reaction with laccase, oxygen needs to be present, and when using peroxidase, if necessary, hydrogen peroxide can be added to the reaction mixture. When setting the reaction temperature, the temperature optimum and the temperature stability of the enzyme to be used can be taken into account.

The amount of enzyme required to cross-link an amount of 1 gram of protein is usually several milligrams, but can be much lower for certain enzymes. For example, in the case a laccase of high purity is used an amount of enzyme of approximately 0.01 to approximately 100 U/ml reaction mixture suffices. Preferably, in that case, an amount of enzyme of approximately 1 to approximately 10 U/ml reaction mixture is used, which corresponds to an amount of preferably 2 to 20 μg enzyme, while as definition it is taken that at an optimal acidity and temperature, 1 U of enzyme catalyses the formation of 1.0 μmol of product per minute. In the case of laccase, this can be determined by following the oxidation of 2,2′-azino-bis-(3-ethylbenzthiazoline-6-sulphonic acid) (ABTS) at 420 nm in the presence of sodium acetate, a pH of 5.0 and 37° C.

The cross-linking reaction can be carried out at a temperature between 5° C. and 80° C., preferably between 35° C. and 40° C.

The cross-linking reaction can be carried out for a period varying from a few minutes to a few days. Preferably, a cross-linking reaction is carried out for a period of between 10 minutes and 24 hours.

The amount of enzyme used, the duration and the temperature during the reaction and the pH of the reaction mixture are not limitative. Depending on the desired end result, for instance the extent of cross-linking, the desired physical property of the cross-linked protein, and the period of time within which it is desired that this be achieved, the temperature of the reaction mixture and the time of cross-linking can be adjusted.

Also, the DP of the phenolic saccharide affects the rate of protein cross-linking, with the lower DPs (e.g. DP 2-9) causing the highest rate of protein-protein (homo) cross-link formation.

A reaction for enzymatic cross-linking of a protein in the presence of a phenolic saccharide in a method of the present invention is aimed at achieving protein-protein cross-links (homo cross-links). The formation of cross-links between the protein and the phenolic saccharide (hetero cross-links) is essentially avoided. The present inventors have found that the DP of the phenolic saccharide influences the ratio between homo and hetero cross-link formation and that more homo cross-links are formed when phenolic oligosaccharides in stead of phenolic polysaccharides are used. In fact, essentially only homo cross-links are formed when the DP of the phenolic saccharide is low. Therefore, preferred embodiments of the invention include the use of a phenolic oligosaccharide as the phenolic saccharide. Preferably a phenolic oligosaccharide with a DP less than 20, preferably less than 10, more preferably less than 6 is used.

Besides performing the reaction in a separate reaction mixture as described in detail hereinabove, the enzymatic cross-linking reaction of a method according to the invention may be performed in situ. Such in situ reactions may involve a reaction that occurs during the normal production process of a foodstuff in order to improve the properties of the proteins comprised therein. Such an in situ method may be performed for instance by addition of a phenolic saccharide (for instance oligomeric pectin hydrolysate as described in more detail above) together with a suitable cross-linking enzyme (for instance laccase or another cross-linking oxidase as described in more detail above) to a protein-comprising product, such as for instance a food product, in amounts sufficient to achieve protein-protein cross-linking within said product.

In yet a further alternative embodiment of an in situ cross-linking method, a suitable cross-linking enzyme may be added to a protein-comprising product and the phenolic saccharide may be generated in situ, for instance by adding e.g. pectin and an enzyme, or enzyme mixture, that is capable of hydrolyzing said pectin into phenolic saccharides. Thus the present invention also provides for protein cross-linking in situ, in the foodstuff wherein the cross-linking is achieved according to this invention by adding to the foodstuff, or by incorporating into the material for preparing the foodstuff, a phenolic polysaccharide (for example sugar beet pectin) and an enzyme capable of hydrolyzing said phenolic polysaccharide (for example the enzyme Driselase) and a cross-linking enzyme (such as laccase). The resulting in situ formation of low DP phenolic saccharides (e.g. monosaccharides and oligosaccharides) will cause or enhance cross-linking of proteins contained in the food product. This process is herein also referred to as in situ enzymatic formation of phenolic saccharide.

The degree of cross-linking attained by a method of the invention can be verified by any method available to the skilled person. Very convenient methods make use of the increase in molecular weight of the resulting reaction product. The molecular weight may for instance conveniently be determined by using electrophoretic methods. A very suitable method is SDS-PAGE, which method is well known in the art of protein science.

In another aspect, the present invention relates to a cross-linked protein obtainable by performing a method of the invention. The cross-linked proteins obtained by the method of the invention exhibit a protein network with interesting functional properties, relevant for application in food products for purposes, such as gelling, foam stabilisation, emulsification, etc. A characteristic of a cross-linked protein of the invention is that it comprises essentially only protein-protein (homo) cross-links.

In another aspect, the present invention relates to a cross-linked protein wherein said protein is cross-linked in the presence of a phenolic saccharide. The details on the phenolic saccharide are as described above for other aspects of the invention.

In yet another aspect, the present invention relates to the use of a phenolic saccharide in a method for enzymatic cross-linking of proteins. Preferably, the use of a phenolic saccharide in a method for enzymatic cross-linking of proteins is related to the enzymatic cross-linking of unmodified globular proteins, and preferably involves a method for cross-linking wherein essentially only protein-protein cross-links are formed. Preferably, in such use the phenolic saccharide has a DP less than 10, preferably less than 6.

In yet another aspect, the present invention relates to the use of a cross-linked protein of the present invention in a foodstuff. In particular, such use will include the improvement of the quality of the food product, such as an improvement of the stability and/or structure of beer foam, improved gelling properties of various food products (especially deserts), strengthening protein networks (gluten), binding together of pieces of meat (restructuring), whey retention (cheese production, e.g. as described in EP 1 057 411), improved melt down resistance (ice cream), fat replacer properties (margarine, mayonnaise, sauces, etc.), consistency/mouth feel properties (sausages, hams, other meat products, tofu, noodles, etc.), coacervates (flavour release, nutrition encapsulation, e.g. as described in EP 0 856 355), etc.

The products herein described may for instance be prepared by using a cross-linked protein of the invention as an ingredient and performing an otherwise conventional preparation process therewith. Alternatively, the products comprising a cross-linked protein of the invention may be prepared by the in situ process as described above, wherein the method of the invention comprises an integral part of the preparation process of said product and wherein the cross-linking of the protein occurs in situ thereby forming the product.

The products of the invention comprising a cross-linked protein of the invention may take any form and may be food products or non-food products

Exemplary food products that may be produced by methods of the present invention may for instance be found in U.S. Pat. No. 5,156,956, U.S. Pat. No. 5,279,839. The skilled person will be able to adapt the methods described in such references in such a way that the cross-linking process is performed as described herein. The methods and cross-linked proteins of the present invention may be used in various baking applications in order to improve the finished product. As an ingredient in meat or a meat product, the cross-linked proteins of the invention may improve the structure of the meat or meat product. Also the gelling properties of protein ingredients used in a meat product may be improved, or equally strong gels may be produced by using less material. These favourable gelling properties may of course be used in any food or non-food application.

In yet another aspect, the present invention relates to a foodstuff comprising a cross-linked protein of the present invention. Examples of foodstuffs that may comprise a cross-linked protein of the present invention are, without limitation, gelled or gellable deserts, foams and foam forming products, such as beer, dairy products such as cheese, spreads or fat replacers such as margarine and mayonnaise, sauces, composite and non-composite meat products such as hams and sausages, ice creams, noodles, tofu, puddings, custards, bread, spreads, toppings, dressings, yogurts, frozen desserts, icings, sour creams, bakery creams, batters, batter coatings, baked products, creamers, shortenings, baby foods, powdered soups, liquid soups and breadings.

The invention may equally suitably be applied to non-food applications, such as for the production of or use in composite materials, the treatment of wool (see e.g. WO 99/60200), silk or other proteinaceous fibres. Other non-food applications include for instance coating processes and coatings themselves that comprise proteins, such as e.g. photographic coatings or paper coatings. Further non-food applications include pharmaceutical or cosmetical applications.

A method of the invention may for instance also be used to improve the characteristics of proteins, such as improving the bloom number of gelatin.

The uses according to these aspects of the invention may include those features as outlined above for a method of the present invention. Preferred proteins and preferred phenolic saccharides are as described above.

### EXAMPLES

### Comparative Example 1

### Cross-Linking of β-Lactoglobulin with Laccase

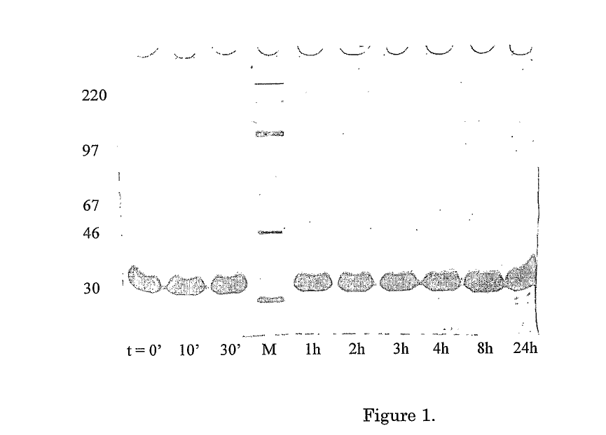

β-Lactoglobulin was purified from whey protein isolate (WPI) using established chromatography methods. An amount of 0.1 gram of β-lactoglobulin was solubilized in 10 ml of 0.1 M phosphate buffer pH 6.0. One milligram of laccase (Trametes versicolor, Wacker Chemie, Munich, Germany) was added and the solution incubated at 40° C. during 24 hours. Samples were taken during the reaction and subjected to SDS-polyacrylamide gel electrophoresis to observe increase in molecular weight of substrate protein, thereby judging results of protein cross-linking. As shown in FIG. 1, almost no cross-linking occurs. Only after incubation for 4 hours or more, a very small fraction of β-lactoglobulin has been converted into the dimeric form. This confirms that a globular protein like β-lactoglobulin in the native state cannot or only to a very small extent be cross-linked enzymatically.

### Comparative Example 2

### Cross-Linking of β-Lactoglobulin with Laccase in the Presence of Ferulic Acid

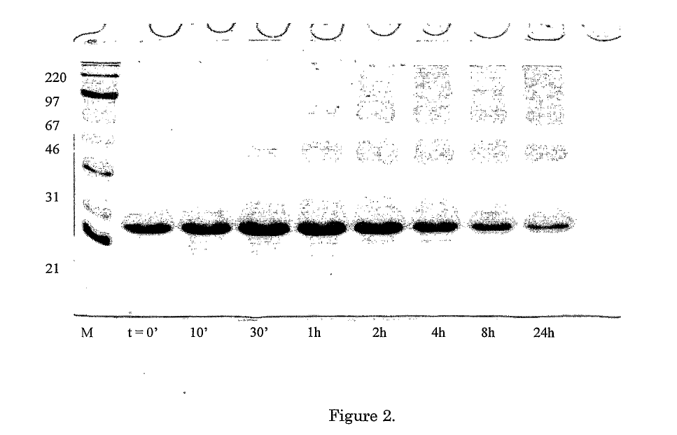

β-Lactoglobulin (0.1 gram) was solubilized in 10 ml of 0.1 M phosphate buffer pH 6.0. One milligram of laccase (Trametes versicolor, Wacker Chemie) was added and the solution incubated at 40° C. during 24 hours. During the first 200 minutes of the reaction, a linear gradient of a decreasing concentration of ferulic acid (Sigma F9500) and an increasing concentration of laccase was added to the reaction. During the 200 minutes, in total 8 milligrams of ferulic acid and 2 milligrams of laccase in 20 ml of 0.1 M phosphate buffer pH 6.0 were added to the reaction mixture. Samples were taken during the reaction and subjected to SDS-polyacrylamide gel electrophoresis to observe increase in molecular weight of substrate protein, thereby judging results of protein cross-linking. The results are shown in FIG. 2. These results show that the molecular weight increases during the reaction and that oligomeric forms of β-lactoglobulin are being formed. After completion of the reaction, most of the monomeric form of β-lactoglobulin has disappeared and oligomeric forms up to a degree of polymerization of approximately 10 are formed. Thus, this Example demonstrates that β-lactoglobulin may be cross-linked in this manner, but that the degree of polymerization of the product is rather low.

### Example 1

### Enzymatic Hydrolysis of Beet Pectin

An amount of 2 grams of sugar beet pectin (Danisco) was solubilized in 100 ml of milliQ water and 200 mg of Driselase (Sigma D9515) was added. Driselase contains various exo- and endo-carbohydrases and glycosidases, including arabinase, galactanase and polygalacturonase, but is devoid of ferulic acid esterase activity. The solution was incubated at 40° C. and the pH was kept constant at pH 4.5 using a pH stat. The reaction was stopped after 2 hours by heating the solution at 80° C. for 10 minutes. The product obtained was called Pectin Hydrolysate 1. The reaction was repeated, but this time the reaction was stopped after 16 hours. The product obtained was called Pectin Hydrolysate 2.

The average degree of polymerization was determined from the reducing power of the products obtained using the Nelson method. Neutral sugar content was determined using Anthron and charged sugars using Blumenkranz.

| Product | Degree of polymerization |
|---|---|
| Pectin Hydrolysate 1 | 5.3 |
| Pectin Hydrolysate 2 | 3.0 |

### Example 2

### Purification of Ferulic Acid-Containing Oligosaccharides from Beet Pectin

Pectin Hydrolysates 1 and 2 as obtained in Example 1 were applied onto a Sepharose™ Fast Flow column (Amersham Biosciences, Buckinghamshire, England), in order to separate charged from uncharged oligosaccharides. The fraction of uncharged oligosaccharides (oligosaccharides that did not bind to the column material) was freeze-dried. The product obtained from Pectin Hydrolysate 1 was called Pectin Oligo 1 and the product obtained from Pectin Hydrolysate 2 was called Pectin Oligo 2.

### Example 3

### Cross-Linking of β-Lactoglobulin with Laccase in the Presence of Feruloyl-Arabinose

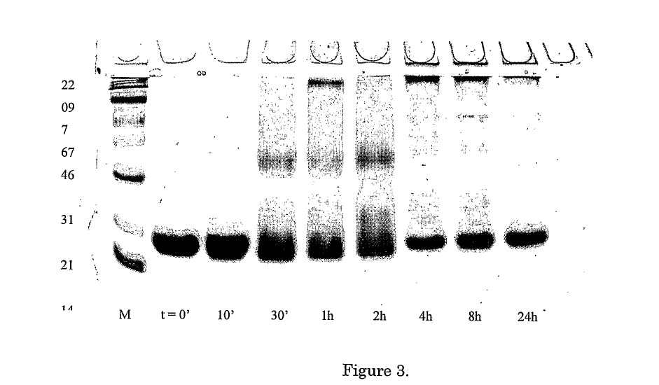

β-Lactoglobulin (0.1 gram) was solubilized in 10 ml of 0.1 M phosphate buffer pH 6.0. One milligram of laccase (Trametes versicolor, Wacker Chemie) was added and the solution incubated at 40° C. during 24 hours. During the first 200 minutes of the reaction, a linear gradient of a decreasing concentration of feruloyl-arabinose and an increasing concentration of laccase was added to the reaction. During the 200 minutes, in total 100 milligrams of feruloyl-arabinose and 2 milligrams of laccase in 20 ml of 0.1 M phosphate buffer pH 6.0 were added to the reaction mixture. Samples were taken during the reaction and subjected to SDS-polyacrylamide gel electrophoresis to observe increase in molecular weight of substrate protein, thereby judging results of protein cross-linking. The results are shown in FIG. 3. These results show that the molecular weight increases during the reaction and that oligomeric forms of β-lactoglobulin are being formed. After completion of the reaction, most of the monomeric form of β-lactoglobulin has disappeared and oligomeric forms with a high degree of polymerization are formed.

### Example 4

### Cross-Linking of β-Lactoglobulin with Laccase in the Presence of Ferulic Acid-Containing Oligosaccharides

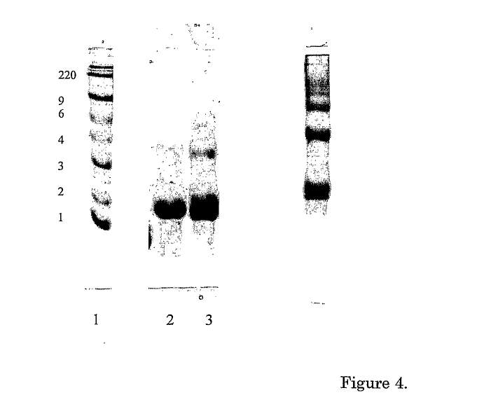

β-Lactoglobulin (0.01 gram) was solubilized in 1 ml of 0.1 M phosphate buffer pH 6.0. 0.1 milligram of laccase (Trametes versicolor, Wacker Chemie) was added and the solution incubated at 40° C. during 10 minutes. 5 mg of Pectin Oligo 1 (obtained from Example 2) was added to the reaction mixture and the solution was incubated for 10 minutes. The same reaction was conducted with Pectin Oligo 2 and reaction products were analysed by SDS-polyacrylamide gel electrophoresis. The results are shown in FIG. 4.

| Lane | Description |
|---|---|
| 1 | Molecular weight markers |
| 2 | β-lactoglobulin |
| 3 | β-lactoglobulin + pectin oligo 1 + laccase |
| 4 | β-lactoglobulin + pectin oligo 2 + laccase |

### Example 5

### Cross-Linking of β-Lactoglobulin with Laccase in the Presence of Pectin Hydrolysate

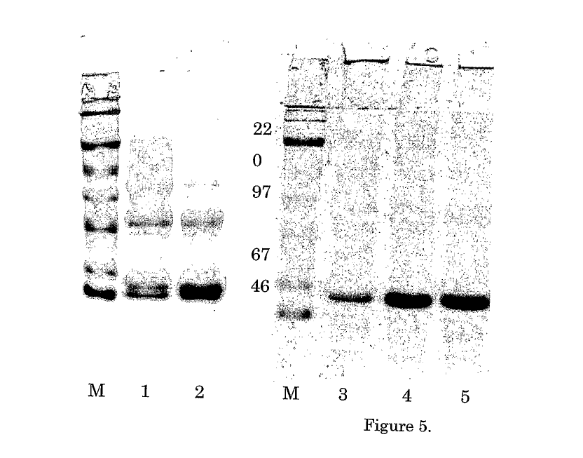

β-Lactoglobulin (0.01 gram) was solubilized in 1 ml of 0.1 M phosphate buffer pH 6.0. 0.1 milligram of laccase (Trametes versicolor, Wacker Chemie) was added and the solution incubated at 40° C. during 10 minutes. 40 mg of hydrolysate 2 (obtained from Example 1) was added to the reaction mixture and the solution was incubated for 10 minutes. The same reaction was conducted with 4 mg of hydrolysate 2. Next, β-Lactoglobulin (0.05 gram) was solubilized in 1 ml of 0.1 M phosphate buffer pH 6.0. 0.1 milligram of laccase was added and the solution incubated at 40° C. during 10 minutes. 150 mg of hydrolysate 2 was added to the reaction mixture and the solution was incubated for 10 minutes. The same reaction was conducted with 75 mg and 38 mg of hydrolysate 2. Reaction products were analysed by SDS-polyacrylamide gel electrophoresis and the results are shown in FIG. 5.

| Lane | Description |
|---|---|
| M | Molecular weight markers |
| 1 | 1% β-lactoglobulin + 40 mg hydrolysate |
| 2 | 1% β-lactoglobulin + 4 mg hydrolysate |
| 3 | 5% β-lactoglobulin + 150 mg hydrolysate |
| 4 | 5% β-lactoglobulin + 75 mg hydrolysate |
| 5 | 5% β-lactoglobulin + 38 mg hydrolysate |

### Example 6

### Permeability of Heat-Induced Protein Gels

### Production of Protein Samples

5.0 gram whey protein isolate BiPro (WPI; Davisco International Inc., La sueur, USA) was solubilized in 100 ml of 0.1 M phosphate buffer pH 6.0, resulting in protein sample “WPI”.

5.0 gram WPI was solubilized in 100 ml of 0.1 M phosphate buffer pH 6.0 and 10 milligram laccase (Trametes versicolor, Wacker Chemie) was added. The solution was incubated at 40° C. for 16 hours, resulting in protein sample “cross-linked WPI”. 5.0 gram WPI was solubilized in 100 ml of 0.1 M phosphate buffer pH 6.0 and 10 milligram of laccase was added. The solution was incubated at 40° C. for 16 hours. During the first 200 minutes of the reaction, a linear gradient of a solution containing a decreasing concentration of feruloyl-arabinose (FA-Ara) and an increasing concentration of laccase was added to the reaction. During the 200 minutes, in total 20 milligram FA-Ara and 2 milligram of laccase were added to the reaction, resulting in protein sample “WPI×FA-Ara low”.

5.0 gram WPI was solubilized in 100 ml of 0.1 M phosphate buffer pH 6.0 and 10 milligram of laccase was added. The solution was incubated at 40° C. for 16 hours. During the first 200 minutes of the reaction, a linear gradient of a solution containing a decreasing concentration of feruloyl-arabinose (FA-Ara) and an increasing concentration of laccase was added to the reaction. During the 200 minutes, in total 100 milligram FA-Ara and 2 milligram of laccase were added to the reaction, resulting in protein sample “WPI×FA-Ara high”.

### Permeability of Heat-Induced Protein Gels

Protein samples were diluted to obtain a final concentration of 4% (w/v), followed by heating at 85° C. for 45 minutes in open-end glass tubes in order to induce gelation. Permeability of heat-induced protein gels was determined at room temperature according to the method of van Dijk and Walstra [Neth. Milk Dairy J. (1986) 40, 3-30]. Low permeability is reflected by a low permeability coefficient.

| | Permeability coefficient B |
|---|---|
| sample | (10⁻¹⁵ m²) |
| WPI | 45 |
| Cross-linked WPI | 18 |
| WPI × FA-Ara low | 10 |
| WPI × FA-Ara high | 5 |

1. A method for enzymatic cross-linking of a protein comprising performing a reaction for enzymatic cross-linking of said protein in the presence of a phenolic saccharide, wherein said reaction comprises the step of forming protein-protein cross-links.

2. Method according to claim 1, wherein said step of forming protein-protein cross-links is performed in the presence of a phenolic saccharide which enhances the rate of a protein-protein cross-linking reaction.

3. Method according to claim 1, wherein said step of forming protein-protein cross-links further comprises essentially avoiding the formation of cross-links between the protein and the phenolic saccharide.

4. Method according to claim 1, wherein said phenolic saccharide is an oligosaccharide or monosaccharide.

5. Method according to claim 1, wherein said phenolic saccharide comprises a saccharide chain length of 1-20, preferably of 1-9, more preferably of 1-5 sugar residues.

6. Method according to claim 1 wherein said phenolic saccharide has a DP less than 10, more preferably less than 9, even more preferably less than 8, still more preferably less than 7, and yet still more preferably less than 6, 5, 4 or 3.

7. Method according to claim 1, wherein said phenolic saccharide is selected from the group consisting of hydrolyzed arabinoxylan, hydrolyzed pectin, p-coumaroylglucose, feruloylglucose and feruloylarabinose.

8. Method according to claim 1, wherein said phenolic saccharide is selected from the group consisting of hydrolyzed sugar beet pectin and feruloylarabinose.

9. Method according to claim 1, wherein said protein is of animal origin, preferably derived from milk.

10. Method according to claim 1, wherein said protein is a globular protein.

11. Method according to claim 1, wherein said protein is a globular protein selected from the group consisting of β-lactoglobulin, α-lactalbumin and bovine serum albumin.

12. Method according to claim 1, wherein the enzyme for performing said enzymatic cross-linking reaction is laccase, a catechol oxidase, a peroxidase or a tyrosinase.

13. Method according to claim 12 wherein the enzyme is laccase.

14. Method according to claim 1 wherein the phenolic saccharide is added to the reaction mixture in one step.

15. Method according to claim 14 wherein the phenolic saccharide is hydrolysed sugar beet pectin.

16. Method according to claim 1 wherein the cross-linking is performed in situ.

17. Method according to claim 1 comprising the in situ enzymatic formation of phenolic saccharide.

18. A cross-linked protein obtainable by a method according to claim 1.

19. A cross-linked protein comprising essentially only protein-protein (homo) cross-links wherein said protein is cross-linked by the action of a phenolic saccharide.

20. Cross-linked protein according to claim 19, wherein said phenolic saccharide is an oligosaccharide or monosaccharide.

21. Cross-linked protein according to claim 19, wherein said phenolic saccharide comprises a saccharide chain length of 1-20, preferably of 1-9, more preferably of 1-5 sugar residues.

22. Cross-linked protein according to claim 19, wherein said phenolic saccharide is selected from the group consisting of hydrolyzed arabinoxylans, hydrolyzed pectins, p-coumaroylglucose, feruloylglucose and feruloylarabinose.

23. Cross-linked protein according to claim 19, wherein said protein is of animal origin, preferably derived from milk.

24. Cross-linked protein according to claim 19-, wherein said protein is a globular protein, preferably selected from the group consisting of β-lactoglobulin, α-lactalbumin, bovine serum albumin and ovalbumin.

25. Use of a phenolic saccharide for enzymatic cross-linking of a protein to enhance the rate of a protein-protein cross-linking reaction.

26. Use of a phenolic saccharide in a method for enzymatic cross-linking of proteins, wherein said phenolic saccharide supports the formation of protein-protein (homo) cross-links.

27. Use according to claim 25, wherein said phenolic saccharide is an oligosaccharide or monosaccharide.

28. Use according to claim 25, wherein said phenolic saccharide comprises a saccharide chain length of 1-20, preferably of 1-9, more preferably of 1-5 sugar residues.

29. Use according to claim 25, wherein said phenolic saccharide has a DP less than 10, more preferably less than 9, even more preferably less than 8, still more preferably less than 7, and yet still more preferably less than 6, 5, 4 or 3.

30. Use according to claim 25, wherein said phenolic saccharide is selected from the group consisting of hydrolyzed arabinoxylan, hydrolyzed pectin, p-coumaroylglucose, feruloylglucose and feruloylarabinose.

31. Use according to claim 30, wherein said phenolic saccharide is selected from the group consisting of hydrolyzed sugar beet pectin and feruloylarabinose.

32. Use according to claim 25, wherein said protein is of animal origin, preferably derived from milk.

33. Use according to claim 25, wherein said protein is a globular protein.

34. Use according to claim 33, wherein said protein is a globular protein selected from the group consisting of β-lactoglobulin, α-lactalbumin and bovine serum albumin.

35. Use according to claim 25, wherein the enzyme for performing said enzymatic cross-linking reaction is laccase, a catechol oxidase, a peroxidase or a tyrosinase.

36. Use according to claim 35, wherein the enzyme is laccase.

37. Use of a cross-linked protein according to claim 18 in a foodstuff, preferably a foodstuff selected from the group consisting of beer foam, puddings, custards, bread, cheeses, ice cream, spreads, toppings, margarine, mayonnaise, sauces, dressings, sausages, hams, other meat products, tofu, noodles, yogurts, frozen desserts, icings, sour creams, bakery creams, batters, batter coatings, baked products, creamers, shortenings, baby foods, powdered soups, liquid soups and breadings.

38. Product comprising a cross-linked protein according to claim 18, preferably a product selected from a coacervate, a gel, a coating or a proteinaceous fibre comprising product.

39. Method according to claim 2, wherein:
said phenolic saccharide is an oligosaccharide or monosaccharide;
said phenolic saccharide comprises a saccharide chain length of 1-20, preferably of 1-9, more preferably of 1-5 sugar residues;
said phenolic saccharide has a DP less than 10, more preferably less than 9, even more preferably less than 8, still more preferably less than 7, and yet still more preferably less than 6, 5, 4 or 3;
said phenolic saccharide is selected from the group consisting of hydrolyzed arabinoxylan, hydrolyzed pectin, p-coumaroylglucose, feruloylglucose and feruloylarabinose;
said phenolic saccharide is selected from the group consisting of hydrolyzed sugar beet pectin and feruloylarabinose;
said protein is of animal origin, preferably derived from milk;
said protein is a globular protein;
said protein is a globular protein selected from the group consisting of β-lactoglobulin, α-lactalbumin and bovine serum albumin;
the enzyme for performing said enzymatic cross-linking reaction is laccase, a catechol oxidase, a peroxidase or a tyrosinase;
the enzyme is laccase;
the phenolic saccharide is added to the reaction mixture in one step;
the phenolic saccharide is hydrolysed sugar beet pectin;
the cross-linking is performed in situ;
and further comprising the in situ enzymatic formation of phenolic saccharide.

40. A cross-linked protein obtainable by a method according to claim 39.

41. Cross-linked protein according to claim 20, wherein:
said phenolic saccharide comprises a saccharide chain length of 1-20, preferably of 1-9, more preferably of 1-5 sugar residues;
said phenolic saccharide is selected from the group consisting of hydrolyzed arabinoxylans, hydrolyzed pectins, p-coumaroylglucose, feruloylglucose and feruloylarabinose;
said protein is of animal origin, preferably derived from milk;
said protein is a globular protein, preferably selected from the group consisting of β-lactoglobulin, α-lactalbumin, bovine serum albumin and ovalbumin.

42. Use according to claim 26, wherein:
said phenolic saccharide is an oligosaccharide or monosaccharide;
said phenolic saccharide comprises a saccharide chain length of 1-20, preferably of 1-9, more preferably of 1-5 sugar residues;
said phenolic saccharide has a DP less than 10, more preferably less than 9, even more preferably less than 8, still more preferably less than 7, and yet still more preferably less than 6, 5, 4 or 3;
said phenolic saccharide is selected from the group consisting of hydrolyzed arabinoxylan, hydrolyzed pectin, p-coumaroylglucose, feruloylglucose and feruloylarabinose;
said phenolic saccharide is selected from the group consisting of hydrolyzed sugar beet pectin and feruloylarabinose;
said protein is of animal origin, preferably derived from milk;
said protein is a globular protein;
said protein is a globular protein selected from the group consisting of β-lactoglobulin, α-lactalbumin and bovine serum albumin;
the enzyme for performing said enzymatic cross-linking reaction is laccase, a catechol oxidase, a peroxidase or a tyrosinase;
the enzyme is laccase.

43. Use of a cross-linked protein according to claim 19 in a foodstuff, preferably a foodstuff selected from the group consisting of beer foam, puddings, custards, bread, cheeses, ice cream, spreads, toppings, margarine, mayonnaise, sauces, dressings, sausages, hams, other meat products, tofu, noodles, yogurts, frozen desserts, icings, sour creams, bakery creams, batters, batter coatings, baked products, creamers, shortenings, baby foods, powdered soups, liquid soups and breadings.

44. Product comprising a cross-linked protein according to claim 19, preferably a product selected from a coacervate, a gel, a coating or a proteinaceous fibre comprising product.
